(19) United States
(12) Patent Application Publication
(10) Pub. No.: US 2014/0101812 A1
(43) Pub. Date: Apr. 17, 2014

(54) SYSTEM AND METHOD FOR EYE PROTECTION

(71) Applicant: COREY RICHARDS, Franklin, ID (US)

(72) Inventor: COREY RICHARDS, Franklin, ID (US)

(21) Appl. No.: 13/649,412

(22) Filed: Oct. 11, 2012

(51) Int. Cl. A61F 9/06

(52) U.S. Cl. 2/12

(57) ABSTRACT

A system and method of eye protection during a welding process comprising the steps of: fabricating a lens, where the lens includes a first degree of shading where the first degree of shading provides a first level of eye protection and a second degree of shading, where the second degree of shading provides a second level of protection, wherein the second level of eye protection shields a user's eye from welding flash rays; inserting photochromic sensing technology in the lens, where the photochromic sensing technology detects welding flash rays; and enabling the second degree of shading of the lens upon the detection of welding flash rays. Further, the second degree of shading continues while the lens is exposed to welding flash rays.

### BACKGROUND OF THE INVENTION

1. Field of Invention

The present invention relates to a system and method for eye protection during welding processes.

2. Description of Related Art

Oxy-fuel cutting and welding is a common process used in the metal fabrication industries. Metal welding or cutting processes include the use fuel gases and oxygen to weld and cut metals. Any worker who performs any metal welding or cutting must wear adequate eye protection in order to protect their eyes from the ultra-violet, infrared and blue light associated welding, commonly known as welding flash. Unprotected eyes exposed to welding flash can become seriously strain and/or damaged. As consequence of this serious danger of injury, eye protection has been specifically designed to protect a worker's eyes from the ultraviolet, infrared and blue light associated with welding flash, according to ANSI Z87-1989 safety standards for a Special Purpose Lens. Any lack of protection from impact, ultra-violet, infrared and blue light may cause severe eyestrain and eye damage. Today very good eye protection can be found designed especially for gas-welding aluminum that cuts the sodium orange flare completely and provides the necessary protection from ultraviolet, infrared and blue light.

### SUMMARY OF THE INVENTION

The present invention relates to a system and method of eye protection during a welding process comprising the steps of: fabricating a lens, where the lens includes a first degree of shading where the first degree of shading provides a first level of eye protection and a second degree of shading, where the second degree of shading provides a second level of protection, wherein the second level of eye protection shields a user's eye from welding flash rays; inserting photochromic sensing technology in the lens, where the photochromic sensing technology detects welding flash rays; and enabling the second degree of shading of the lens upon the detection of welding flash rays. Further, the second degree of shading continues while the lens is exposed to welding flash rays.

### BRIEF DESCRIPTION OF DRAWINGS

FIG. 1 depicts an eye protection system according to the present invention for use during exposure to ultraviolet, infrared and blue light associated with welding flash.

### DETAILED DESCRIPTION

The present invention relates to a system and method of eye protection for use while performing welding. The present invention provides a fabricated safety lens that provides a shield to protect a user's eyes from welding flash rays. In one particular embodiment, the eye protection lens may be utilized in an eyeglass frame and have sufficient circumference to cover the eyes from rays that are associated with welding. The lens in accordance with the present invention includes a first degree of shading that provides a first level eye protection and then a second degree of shading that provides a second degree of eye protection wherein the second degree of eye protection is utilized to protect the eyes from welding flash rays. The fabricated lens further includes a sensing technology that detects welding flash rays and enables the darker or second degree of shading upon the detection of the welding flash. The lenses in accordance with the present invention provide the user with convenient lenses that may be worn continuously and avoids the repetitive flipping of a hood or the flipping of a lens over the eyes of a user. Such flipping is common among welders that typically use a hooded facemask that includes a flash lens.

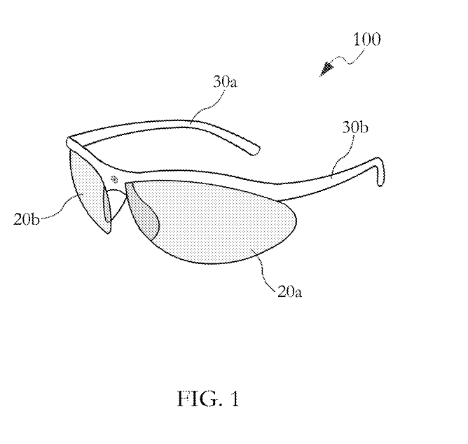

In reference to FIG. 1, an exemplary embodiment of the present invention is depicted. FIG. 1 includes eyeglass frame assembly frame assembly 100 that incorporates welding flash lenses 20a, 20b. The eyeglass frame 100 also includes temples 30a, 30b. The frame assembly 100 includes nose pads provided at the bridge of the eyeglass assembly.

These lenses 20a, 20b include photochromic sensing technology that activates a shade changing feature of the lenses. The lenses are photosensitive in that a certain degree of shading is provided with the lenses during normal use that creates the first degree of shading in accordance with the present invention. The photosensitivity of the lenses reacts upon exposure to the infrared and blue light associated with the welding flash. The lenses darken to the second degree shading to protect the user's eye from the welding flash that is associated with the welding processes. Consequently the glasses perform a function of eye protection wherein a first degree of shading is provided for the user and then a second degree of shading is provided upon exposure to welding flash. The change in shading is provided through the use of the photosensitive technology implanted within the lenses.

The lenses in accordance with the present invention provide the user with a convenient means for protecting the eyes while performing welding processes. It is imperative that the eyes are provided with this protection to prevent injuries to the eyes due to the violet and infrared rays that are created while an individual performs welding. Further the use of the photosensitive lenses in accordance with the present invention obviates the need for a welding helmet which requires constant flipping during use by a welder. The instant invention has been shown and described in what it considers to be the most practical and preferred embodiments. It is recognized, however, that departures may be made there from within the scope of the invention and that obvious modifications will occur to a person skilled in the art.

What is claimed is:

1. A method of eye protection during a welding process comprising the steps of:
fabricating a lens, where the lens includes a first degree of shading where the first degree of shading provides a first level of eye protection and a second degree of shading, where the second degree of shading provides a second level of protection, wherein the second level of eye protection shields a user's eye from welding flash rays;
inserting photochromic sensing technology in the lens, where the photochromic sensing technology detects welding flash rays; and
enabling the second degree of shading of the lens upon the detection of welding flash rays.

2. The method of eye protection according to claim 1, where the second degree of shading continues while the lens is exposed to welding flash rays.

3. The method of eye protection according to claim 1, further including the step of disabling the second degree of shading upon removal of welding flash ray exposure; and
returning the lens to the first degree of shading.

4. A eye protection system for protecting a user's eyes while the user is exposed to welding flash rays comprising:
a pair of lens, where the pair of lens include a first degree of shading where the first degree of shading provides a first level of eye protection and a second degree of shading, where the second degree of shading provides a second level of protection, wherein the second level of eye protection shields a user's eye from welding flash rays;
photochromic sensing technology in each lens, where the photochromic sensing technology detects welding flash rays and triggers the second degree of shading of the lens upon the detection of welding flash ray exposure; and
a frame assembly, where the assembly secures the pair of lens for use by the user.

5. The eye protection system according to claim 4, where the pair lens provides the first degree of shading when the lens are not exposed to welding flash rays.
